(19) United States
(12) Patent Application Publication
(10) Pub. No.: US 2013/0096317 A1
(43) Pub. Date: Apr. 18, 2013

(54) REDUCTION OF ALDEHYDES AND KETONES TO ALCOHOLS

(71) Applicants: Charles L. Liotta, Atlanta, GA (US); Pamela Pollet, Atlanta, GA (US); Kristen Kitagawa Fisher, Sammamish, WA (US); William Dubay, Folsom, CA (US); Joy Stringer, Folsom, CA (US)

(72) Inventors: Charles L. Liotta, Atlanta, GA (US); Pamela Pollet, Atlanta, GA (US); Kristen Kitagawa Fisher, Sammamish, WA (US); William Dubay, Folsom, CA (US); Joy Stringer, Folsom, CA (US)

(73) Assignees: GEORGIA TECH RESEARCH CORPORATION, Atlanta, GA (US); AMERICAN PACIFIC CORPORATION, Las Vegas, NV (US)

(21) Appl. No.: 13/509,580

(22) PCT Filed: Nov. 12, 2010
(86) PCT No.: PCT/US10/56592

§ 371 Date: Nov. 29, 2012

(30) Foreign Application Priority Data
Nov. 12, 2009 (US) 61260747

(51) Int. Cl. C07C 269/06; C07C 29/14; C07C 29/143; C07D 209/48; C07C 231/12

(52) U.S. Cl. CPC C07C 269/06; C07D 209/48; C07C 231/12; C07C 29/143; C07C 29/14
USPC 548/479; 560/29; 548/478; 564/213; 564/212; 568/814

(57) ABSTRACT

The embodiments described herein provide a reduction of an aldehyde or a ketone, such as a Meerwein-Ponnorf-Verley (MPV) reaction of an aldehyde or ketone. In some embodiments, the reaction occurs in the presence of Al[OC(CH₃)₃]. In some embodiments, the reaction occurs in the presence of an aprotic solvent. In some embodiments, the aldehyde or ketone is an amino aldehyde or an amino ketone wherein the amine is group is protected such that the nitrogen of the amine has no proton. Other embodiments related to compositions and compounds related to the reduction reaction, or to the preparation or use of the aldehyde, the ketone, or the resulting alcohol.

### BACKGROUND OF THE INVENTION

1. Field of the Invention

The embodiments disclosed herein relate to reduction of an aldehyde or ketone, such as by aluminum alkoxides in the presence of alcohols comprising a carbon atom which is directly bonded to a hydroxyl group and at least one hydrogen atom.

2. Description of the Related Art

The Meerwein-Ponndorf-Verley (MPV) reduction of carbonyl-containing compounds (e.g. aldehydes and ketones) to the corresponding alcohol has often been used because (1) the reaction may use relatively inexpensive reagents, (2) the procedure may be relatively straightforward and may employ relatively mild conditions, and (3) the reaction may be chemoselective. However, more than catalytic amounts of the metal catalyst may be required to achieve high yields in a reasonable amount of time. The MPV reaction is reversible, and the reverse reaction is known as the Oppenauer oxidation. Generally, the MPV reduction is catalyzed by metal alkoxides and utilizes a secondary alcohol as a hydride source. Most often, the reaction is carried out using Al(OiPr)₃ in isopropanol, but other metal reagents, such as lanthanide metals, have also been reported.

Stereoselective MPV reductions have been performed by varying the reaction conditions. One approach, using optically active alcohols as the solvent, has had moderate success in achieving enantioselectivity in the reduction of some prochiral aldehydes and ketones, but the process may be impractical for commercial purposes. Another approach takes advantage of the presence of a chiral center (R or S) promimate to the aldehyde or ketone functionality which is being reduced to the corresponding alcohol. The diasteromeric excess is often modest at best. In addition to the problems which may be associated with the stereoselective MPV reductions of aldehydes and ketones, large quantities of aluminum alkoxides may often be employed to allow the reaction to take place in a reasonable period of time. This may often lead to the production of large quantities of aluminum salt by-produces which may need to be disposed of. This, of course, may potentially be a negative contributor to the environment. Therefore, there is a continuing need to provide efficient, cost-effective, and sustainable reductions via MPV mediated processes. In addition, efficient, cost-effective, and sustainable diastereoselective MPV reductions of aldehydes and ketones containing proximate chiral centers (R or S) are also needed.

### SUMMARY OF THE INVENTION

N-(tert-butyloxycarbonyl)-(3S)-3-amino-1-chloro-4-phenyl-2-butanone (1) and its respective diastereomeric reduction products, N-(tert-butyloxycarbonyl)-(3S)-3-amino-1-chloro-4-phenyl-(2S)-butanol (2) and N-(tert-butyloxycarbonyl)-(3S)-3-amino-1-chloro-4-phenyl-(2R)-butanol (3), are important intermediates in the synthesis of HIV protease inhibitors such as Saquinavir®, Amprenavir®, and Atazanavir® (Scheme). Not only may these drugs be useful in preventing the spread of HIV, but they may also have potential for cancer treatments.

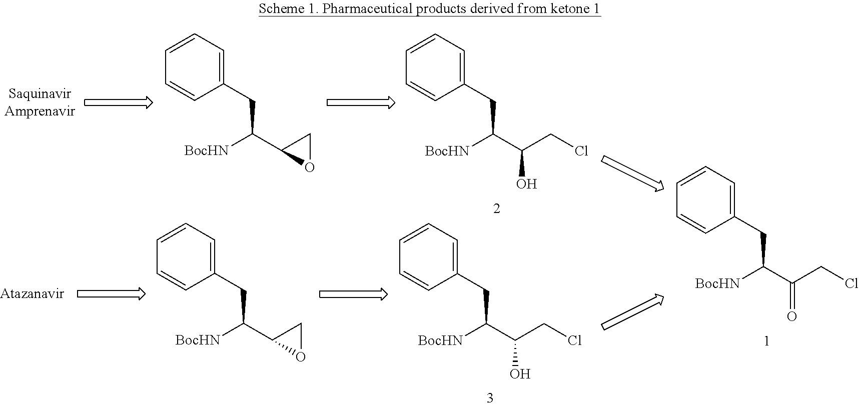 [SMILES: CC(C)(C)OC(=O)N[C@@H](Cc1ccccc1)[C@@H]1CO1 ; CC(C)(C)OC(=O)N[C@@H](Cc1ccccc1)[C@H]1CO1 ; CC(C)(C)OC(=O)N[C@@H](Cc1ccccc1)[C@@H](O)CCl ; CC(C)(C)OC(=O)N[C@@H](Cc1ccccc1)[C@H](O)CCl ; CC(C)(C)OC(=O)N[C@@H](Cc1ccccc1)C(=O)CCl]

The selective preparation of the diastereomers 2 and 3 from the corresponding prochiral ketone 1 has been investigated. These studies have shown that the MPV reduction of 1 using Al(OiPr)₃ in isopropanol proceeds cleanly to yield alcohol 2 in diastereomeric excesses ranging from 94% to 97%. However, the stereoselective synthesis of (R,S) diastereomer 3 may be more challenging. A wide variety of reagents for this reduction have been investigated, including aluminohydride reagents, borohydride reagents, heterogeneous transition metal catalysts, homogeneous chiral catalysts, and emzymatic reductions employing microorganisms. However, the techniques that may produce high yields and stereoselective production of 3 may be expensive for large scale production and the cheaper methods may yield lower selectivity for alcohol 3.

The embodiments described herein provide a reduction of an aldehyde or a ketone, such as a Meerwein-Ponnorf-Verley (MPV) reaction of an aldehyde or ketone, in the presence of an aluminum alkoxide and an alcohol comprising a carbon atom directly bonded to both a hydroxyl group and a hydrogen atom. In some embodiments, the reaction occurs in the presence of Al[OC(CH₃)₃]₃ or other aluminum alkoxides in which the alkoxide portion does not contain a hydrogen attached to the carbon bearing the oxygen-aluminum bond. In these cases an alcohol is present which comprises a carbon atom directly bonded to both a hydroxyl group and a hydrogen atom. In some embodiments, the reaction occurs in the presence of an aprotic solvent. In some embodiments, the aldehyde or ketone is an amino aldehyde or an amino ketone wherein the amine is group is protected. In some embodiments the amine protecting group is such that the protected amine has no proton attached to the nitrogen atom.

Some embodiments provide a method of reducing a C═O of an aldehyde or a ketone to a CH—OH of a product alcohol comprising reacting the aldehyde or ketone in the presence of Al[OC(CH₃)₃]₃ and a reactant alcohol.

Some embodiments relate to a method of reducing a C═O of an aldehyde or a ketone to a CH—OH of a product alcohol comprising reacting the aldehyde or ketone in the presence of: Al(OR^{o})3, a reactant alcohol, and an aprotic solvent, wherein each R^{o} is independently C₁₋₆ alkyl or optionally substituted aryl.

Some embodiments provide a method of reducing a C═O of an aldehyde or a ketone to a CH—OH of a product alcohol wherein the aldehyde or ketone is represented by Formula 1:

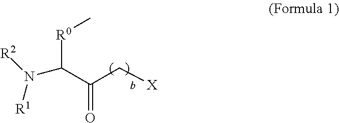

or a salt thereof; wherein X is H, a halogen, R^{a}C(═O)O—, or R^{a}S(═O)₂O—, wherein R^{a} is C₁₋₆ alkyl, C₁₋₆F₁₋₁₃ fluoroalkyl, or optionally substituted phenyl;Y is H, optionally substituted C₆₋₁₀ aryl, optionally substituted C₂₋₁₀ heteroaryl, halo, —OR^{b}, —SR^{b}, —NR^{b}R^{c}, —CO₂R^{b}, —OC(═O)R^{b}, —C(═O)R^{b}, —C(═O)NR^{b}R^{c}, or —NR^{b}—C(═NR^{c})—NR^{d}R^{e}, wherein R^{b}, R^{c}, R^{d}, and R^{e} are independently H, C₁₋₆ alkyl, or a protecting group; R¹ is H, C₁₋₆ alkyl, COR³, or a protecting group, or R¹ and Y may together be a covalent bond connecting R⁰ to the nitrogen atom; R² is H, C₁₋₆ alkyl, COR³, or a protecting group; each R³ is H or optionally substituted C₁₋₁₂ hydrocarbyl; R⁰ is C₁₋₆ alkyl; and b is 0, 1, 2, or 3.

Some embodiments provide a method of reducing a C═O of a ketone to a CH—OH of a product alcohol wherein the ketone is represented by Formula 2:

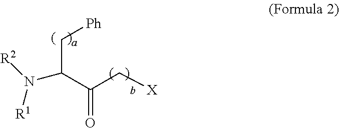

wherein R¹ is COR³; R² is H or COR³; each R³ is H or optionally substituted C₁₋₁₂ hydrocarbyl and X is a halogen, CF₃SO₃—, or Ph¹-SO₃—; Ph and Ph¹ are independently optionally substituted phenyl; a is 0, 1, 2, or 3; and b is 1, 2, or 3.

Some embodiments relate to a composition comprising an aldehyde or a ketone, a product alcohol, Al(OR^{o})₃, and a reactant alcohol; wherein: each R^{o} is C(CH₃)₃; the aldehyde or the ketone and the product alcohol have identical structures except that the aldehyde or ketone has a C═O in the same position occupied by a CH—OH of the product alcohol ; or each R^{o} is C₁₋₆ alkyl; and the ketone is represented by a Formula 2, the product alcohol is represented by Formula 3:

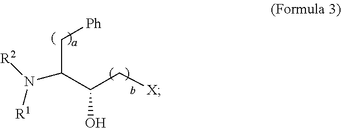

and
R¹, R², Ph, X, a, and b of the ketone are the same as R¹, R², Ph, X, a, and b of the product alcohol; wherein R¹ is COR³; R² is H or COR³; each R³ is H or optionally substituted C₁₋₁₂ hydrocarbyl; and X is a halogen, CF₃SO₃—, or Ph¹-SO₃—; Ph and Ph¹ are independently optionally substituted phenyl; a is 0, 1, 2, or 3; and b is 1, 2, or 3.

Some embodiments provide compound 7 or compound 10. Some embodiments provide a composition comprising compound 7 and compound 107, or a composition comprising compound 10 and compound 110.

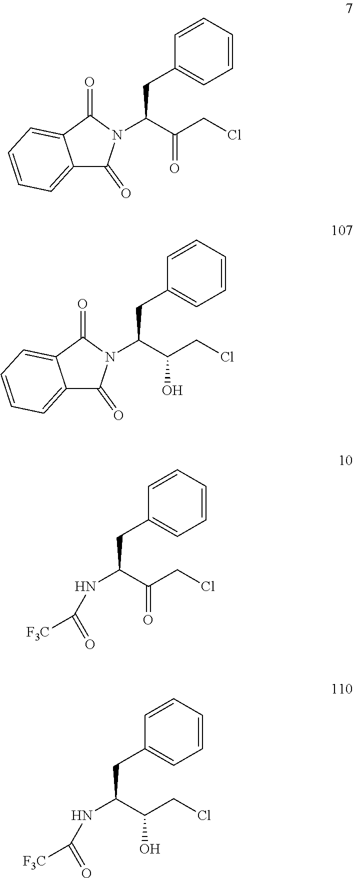 [SMILES: O=C(CCl)[C@H](Cc1ccccc1)N1C(=O)c2ccccc2C1=O ; O=C1c2ccccc2C(=O)N1[C@@H](Cc1ccccc1)[C@@H](O)CCl ; O=C(CCl)[C@H](Cc1ccccc1)NC(=O)C(F)(F)F ; O=C(N[C@@H](Cc1ccccc1)[C@@H](O)CCl)C(F)(F)F]

### BRIEF DESCRIPTION OF THE DRAWINGS

FIG. 1 is a plot of the formation of an alcohol from ketone 1 over time using Al(OiPr)₃ or Al(OtBu)₃ as a catalyst in isopropanol solvent.

FIG. 2 is a plot of the formation of an alcohol from ketone 1 over time using Al(OiPr)₃ or Al(OtBu)₃ as a catalyst and a toluene/isopropanol (9/1 vol/vol) cosolvent system.

FIG. 3 is a plot of the formation of sec-phenylethanol from acetophenone over time using Al(OiPr)₃ or Al(OtBu)₃ as a catalyst in isopropanol solvent.

FIG. 4 is a plot of the formation of sec-phenylethanol from acetophenone over time using Al(OiPr)₃ or Al(OtBu)₃ as a catalyst and a toluene/isopropanol (9/1 vol/vol) cosolvent system.

FIG. 5 is a plot of the formation of benzyl alcohol from benzaldehyde over time using Al(OiPr)₃ or Al(OtBu)₃ as a catalyst in isopropanol solvent.

FIG. 6 is a plot of the formation of benzyl alcohol from benzaldehyde over time using Al(OiPr)₃ or Al(OtBu)₃ as a catalyst and a toluene/isopropanol (9/1 vol/vol) cosolvent system.

### DETAILED DESCRIPTION OF THE PREFERRED EMBODIMENT

The terms “aldehyde,” “ketone,” and “alcohol” have the ordinary meaning understood by a person of ordinary skill in the art.

The term “aprotic solvent” has the ordinary meaning understood by a person of ordinary skill in the art. In some embodiments, the term “aprotic solvent” includes a solvent wherein all of the protons are substantially less acidic than the proton on a hydroxyl moiety. Examples may include ethers, esters, N,N-disubstituted amides, hydrocarbons, etc.

The term “diastereomer” has the ordinary meaning understood by a person of ordinary skill in the art. In some embodiments, the term “diastereomer” includes to a stereoisomer which is not an enantiomer and comprises at least 2 chiral centers. [What does this last sentence mean?]

The term “salt” has the ordinary meaning understood by a person of ordinary skill in the art. In some embodiments, the term “salt” includes one or more ionic forms of a compound, such as a conjugate acid or base, associated with one or more corresponding counter-ions. In some embodiments, salts can form from or incorporate one or more deprotonated acidic groups (e.g. carboxylic acid/carboxylate), one or more protonated basic groups (e.g. amine/ammonium), or both (e.g. zwitterions).

Unless otherwise indicated, when a chemical structural feature such as hydrocarbyl or phenyl is referred to as being “optionally substituted,” it is meant that the feature may have no substituents (i.e. be unsubstituted) or may have one or more substituents. A feature that is “substituted” has one or more substituents. The term “substituent” has the ordinary meaning known to one of ordinary skill in the art. In some embodiments, the substituent is an ordinary organic moiety known in the art, which may have a molecular weight (e.g. the sum of the atomic masses of the atoms of the substituent) of less than: about 500 g/mol, about 300 g/mol, about 200 g/mol, about 100 g/mol, or about 50 g/mol. [g/m ???] In some embodiments, the substituent comprises: about 0-30, about 0-20, about 0-10, or about 0-5 carbon atoms; and about 0-30, about 0-20, about 0-10, or about 0-5 heteroatoms independently selected from: N, O, S, P, Si, F, Cl, Br, I, and combinations thereof; provided that the substituent comprises at least one atom selected from: C, N, O, S, P, Si, F, Cl, Br, and I. Examples of substituents include, but are not limited to, alkyl, alkenyl, alkynyl, carbazolyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, and amino, including mono- and di-substituted amino groups, and the protected derivatives thereof.

As used herein the term “aryl” has the ordinary meaning understood by a person of ordinary skill in the art. In some embodiments, the term “aryl” may refer to an aromatic ring or aromatic ring system such as phenyl, naphthyl, etc. The term “heteroaryl” also has the meaning understood by a person of ordinary skill in the art, and in some embodiments, may refer to an “aryl” which has one or more heteroatoms in the ring or ring system. Examples of “heteroaryl” may include, but are not limited to, pyridinyl, furyl, thienyl, oxazolyl, thiazolyl, imidazolyl, indolyl, quinolinyl, benzofuranyl, benzothienyl, benzooxazolyl, benzothiazolyl, benzoimidazolyl, etc.

As used herein, the term “hydrocarbyl” includes a moiety composed of carbon and hydrogen. Hydrocarbyl includes alkyl (e.g. comprising no double or triple bonds), alkenyl (e.g. comprising at least 1 double bond), alkynyl (e.g. comprising at least 1 triple bond), aryl, etc., and combinations thereof, and may be linear, branched, cyclic, or a combination thereof. Hydrocarbyl may be bonded to any other number of moieties (e.g. be bonded to 1 other group, such as —CH₃, —CH═CH₂, etc.; 2 other groups, such as -phenyl-, —C═C—, etc.; or any number of other groups) that the structure may bear, and in some embodiments, may contain from one to thirty-five carbon atoms. Examples of hydrocarbyl groups include but are not limited to C₁ alkyl, C₂ alkyl, C₂ alkenyl, C₂ alkynyl, C₃ alkyl, C₃ alkenyl, C₃ alkynyl, C₄ alkyl, C₄ alkenyl, C₄ alkynyl, C₅ alkyl, C₅ alkenyl, C₅ alkynyl, C₆ alkyl, C₆ alkenyl, C₆ alkynyl, phenyl, etc.

As used herein, the term “alkyl” includes a moiety composed of carbon and hydrogen containing no double or triple bonds. Alkyl may be linear, branched, cyclic, or a combination thereof, may be bonded to any other number of moieties (e.g. be bonded to 1 other group, such as —CH₃, 2 other groups, such as —CH₂—, or any number of other groups) that the structure may bear, and in some embodiments, may contain from one to thirty-five carbon atoms. Examples of alkyl groups include but are not limited to CH₃ (e.g. methyl), C₂H₅ (e.g. ethyl), C₃H₇ (e.g. propyl isomers such as propyl, isopropyl, etc.), C₃H₆ (e.g. cyclopropyl), C₄H₉ (e.g. butyl isomers) C₄H₈ (e.g. cyclobutyl isomers such as cyclobutyl, methylcyclopropyl, etc.), C₅H₁₁ (e.g. pentyl isomers), C₅H₁₀ (e.g. cyclopentyl isomers such as cyclopentyl, methylcyclobutyl, dimethylcyclopropyl, etc.) C₆H₁₃ (e.g. hexyl isomers), C₆H₁₂ (e.g. cyclohexyl isomers), C₇H₁₅ (e.g. heptyl isomers), C₇H₁₄ (e.g. cycloheptyl isomers), C₈H₁₇ (e.g. octyl isomers), C₈H₁₆ (e.g. cyclooctyl isomers), C₉H₁₉ (e.g. nonyl isomers), C₉H₁₈ (e.g. cyclononyl isomers), C₁₀H₂₁ (e.g. decyl isomers), C₁₀H₂₀ (e.g. cyclodecyl isomers), C₁₁H₂₃ (e.g. undecyl isomers), C₁₁H₂₂ (e.g. cycloundecyl isomers), C₁₂H₂₅ (e.g. dodecyl isomers), C₁₂H₂₄ (e.g. cyclododecyl isomers), C₁₃H₂₇ (e.g. tridecyl isomers), C₁₃H₂₆ (e.g. cyclotridecyl isomers), and the like.

As used herein, the term “fluoroalkyl” includes alkyl having one or more fluoro substituents. The term “perfluoroalkyl” includes fluoroalkyl wherein all hydrogen atom are replaced by fluoro such as —CF₃, —C₂F₅, —C₃F₇, —C₄F₉, etc. An expression such as “C₁₋₁₂” (e.g. “C₁₋₁₂ hydrocarbyl”) refers to the number of carbon atoms in a moiety, and similar expressions have similar meanings. Generally, an expression such as “C₁₋₁₂” (e.g. “C₁₋₁₂ hydrocarbyl”) refers only to the number of carbon atoms in a parent group, and does not characterize or limit the substituents in any way. If there any doubt arises as to whether a structural feature is a substituent or a parent group, the carbon atoms should be counted as if the structural feature is part of the parent group. For example, the carbon atoms of an alkyl “substituent” on an alkyl parent should be counted as part of the parent group.

As used herein the term “protecting group” has the ordinary meaning understood by a person of ordinary skill in the art. In some embodiments, the term “protecting group” includes a group which is stable during a reduction of an aldehyde or ketone to an alcohol, such as in the MPV reduction, but which may later be removed by a deprotection step.

In some embodiments, an amine may be protected by an amide, so that the protecting group is —C(═O)R, wherein R may be, for example, optionally substituted alkyl such as methyl or CF₃, optionally substituted phenyl, trialkylsilyl, H, etc. In some embodiments, an amine may be protected by an imide, such as 2 independent —C(═O)R groups described above, or a divalent moiety attaching to the amine nitrogen in two positions, such as —C(═O)R*—C(═O)—, wherein R* is optionally substituted alkyl or optionally substituted phenyl. In some embodiments, the amine may be protected by a carbamate, so that the protecting group is —C(═O)OR, wherein R is described above. Carbamates such as fluorenylmethyl carbamate also be used. In some embodiments, an amine may be protected by an N-sulfonamide, so that the protecting group is —SO₂R, wherein R is described above. Other protecting groups may also include optionally substituted benzyl, —Si(CH₃)₂—CH₂CH₂Si(CH₃)₂—, —CH₂O(CH₂)₃CH═CH₂, and others known in the art. Further examples of protecting groups that may be used to protect amine functional groups are depicted below, wherein Ph is optionally substituted phenyl.

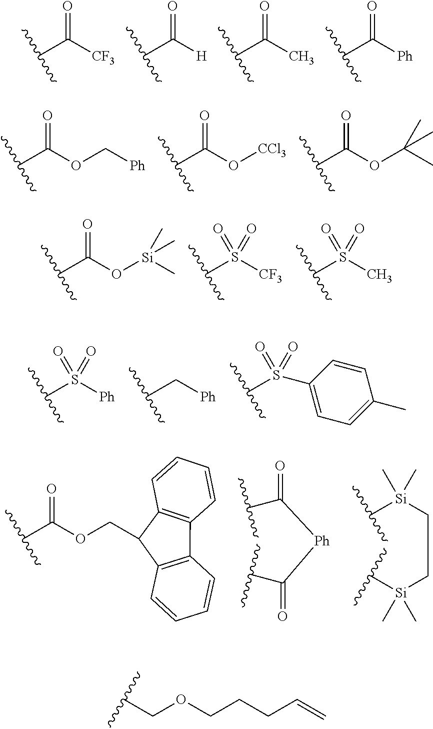

In some embodiments, an alcohol or a thiol may be protected by an ether or a thioether, so that the protecting group is optionally substituted alkyl (such as methyl, t-butyl, optionally substituted triphenylmethyl, etc.), optionally substituted alkoxymethyl (such as methoxymethyl, 1-ethoxyethyl, 2-methoxypropyl, etc.), optionally substituted methyl sulfide such as —CH₂SCH₃; an ester or a thioester, so that the protecting group is acyl, such as acetyl, pivoloyl, benzoyl, etc.; a silyl ether or a silyl thioether, so that the protecting group is a silane, such as trimethylsilane, triethylsilane, triisopropylsilane, triphenylsilane, etc. Further examples of protecting groups that may be used to protect hydroxyl or thiol functional groups are depicted below, wherein Ph is optionally substituted phenyl.

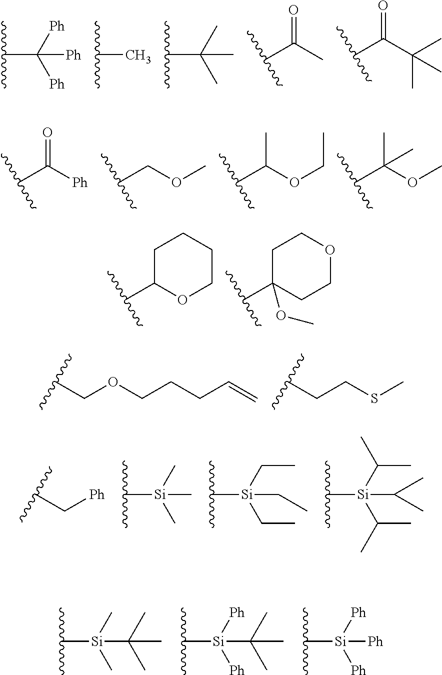

Generally, the methods described herein may be used to reduce a C═O of an aldehyde or a ketone to CH—OH of a product alcohol. In some embodiments, the reduction provided herein is carried out by reacting the aldehyde or a ketone in the presence of an aluminum alkoxide and a reactant alcohol.

Typically, the aldehyde or ketone is reacted in the presence of a catalyst and a reactant alcohol so that a product alcohol is formed. Thus, the aldehyde or the ketone and the product alcohol generally have identical structures except that the aldehyde or ketone has a C═O in the same position occupied by a CH—OH of the product alcohol.

While the reaction is progressing, a composition comprising the aldehyde or ketone and the product alcohol may be formed. A composition comprising the aldehyde or ketone and the product alcohol may also be a product of the reaction if conversion is not complete, which may often be the case. Thus, some embodiments provide a composition comprising a compound of Formula 2 and a compound of Formula 3 wherein R¹, R², Ph, X, a, and b of the compound of Formula 2 are the same as R¹, R², Ph, X, a, and b of the compound of Formula 3. In other words, in these embodiments, the compound of Formula 2 is identical to the compound of Formula 3, except for the C═O of Formula 2 and the CH—OH of Formula 3. For example some embodiments provide a composition comprising compound 7 and compound 107; or a composition comprising compound 10 and compound 110.

Any aldehyde or ketone may be used. In some embodiments, the aldehyde or ketone may further comprise a protected or unprotected amine, a halogen, and/or an optionally substituted phenyl group. In some embodiments, the aldehyde or ketone is represented by Formula 1 (depicted above). In some embodiments, the reaction comprises converting an aldehyde or ketone of Formula 1 to an alcohol of Formula 1a.

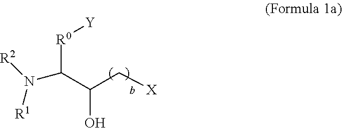

With respect to Formula 1 and Formula 1a, R⁰ is C₁₋₆ alkyl such as linear or branched represented by a formula: —CH₂—, —C₂H₄—, —C₃H₆—, —C₄H₈—, —C₅H₁₀—, —C₆H₁₂—, etc., cyclic alkyl represented by a formula: —C₃H₄—, —C₄H₆—, —C₆H₁₀—, etc. R^{o} may also be a bond. Formula 1b depicts some embodiments of an aldehyde or ketone wherein R^{o} is a bond. Formula 1b depicts some embodiments of a product alcohol wherein R^{o} is a bond.

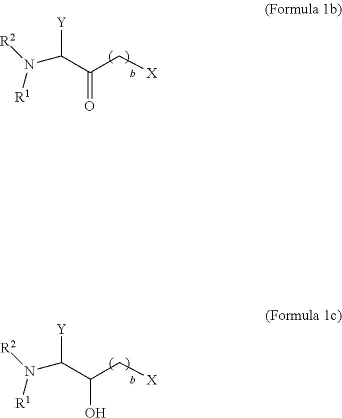

With respect to Formula 1, Formula 1a, Formula 1b, and Formula 1c, Y is H, optionally substituted C₆₋₁₀ aryl such as optionally substituted phenyl; optionally substituted C₂₋₁₀ heteroaryl such as optionally substituted indolyl, optionally substituted imidazolyl, optionally substituted oxazolyl, optionally substituted thiazolyl, etc.; halo such as F, Cl, Br, I, etc.; —OR^{b}, —SR^{b}, —NR^{b}R^{c}, —CO₂R^{f}, —OC(═O)R^{f}, —C(═O)R^{f}, —C(═O)NR^{b}R^{c}, or —NR^{b}—C(═NR^{c})—NR^{d}R^{e}. In some embodiments, R^{b}, R^{c}, R^{d}, R^{e}, and R^{f} are independently H; C₁₋₆ alkyl such as methyl, ethyl, propyl isomers, cyclopropyl, butyl isomers, cyclobutyl isomers, pentyl isomers, cyclopentyl isomers, hexyl isomers, cyclohexyl isomers; or a protecting group.

In some embodiments, the ketone is represented by Formula 2 (depicted above). In some embodiments, the reaction comprises converting a ketone of Formula 2 to an alcohol of Formula 3 (depicted above).

With respect to Formula 1, Formula 1a, Formula 1b, Formula 1 c, Formula 2 and Formula 3, R¹ is H, C₁₋₆ alkyl, COR³, or a protecting group, wherein each R³ is H, or optionally substituted C₁₋₁₂ hydrocarbyl. In some embodiments, R³ may be optionally substituted phenyl or optionally substituted C₁₋₆ alkyl such as methyl, ethyl, propyl isomers, cyclopropyl, butyl isomers, cyclobutyl isomers, pentyl isomers, cyclopentyl isomers, hexyl isomers, cyclohexyl isomers, or the like.

With respect to Formula 1, Formula 1a, Formula 1b, Formula 1c, R¹ and Y may together be a covalent bond connecting R⁰ to the nitrogen atom;

With respect to Formula 1, Formula 1a, Formula 1b, Formula 1c, Formula 2 and Formula 3, R² is H, C₁₋₆ alkyl, COR³, or a protecting group, wherein each R³ is H, or optionally substituted C₁₋₁₂ hydrocarbyl. In some embodiments, R³ may be optionally substituted phenyl or optionally substituted C₁₋₆ alkyl such as methyl, ethyl, propyl isomers, cyclopropyl, butyl isomers, cyclobutyl isomers, pentyl isomers, cyclopentyl isomers, hexyl isomers, cyclohexyl isomers, or the like.

R¹ and R² may together form a ring. For example —NR¹R² may form an optionally substituted succinimide, an optionally substituted phthalimide, etc.

With respect to Formula 1, Formula 1a, Formula 1b, Formula 1c, Formula 2 and Formula 3, X is H; a halogen, R^{a}C(═O)O—, or R^{a}S(═O)₂O—, wherein R^{a} is C₁₋₆ alkyl (such as methyl, ethyl, propyl isomers, cyclopropyl, butyl isomers, cyclobutyl isomers, pentyl isomers, cyclopentyl isomers, hexyl isomers, cyclohexyl isomers, etc.), C₁₋₆F₁₋₁₃ fluoroalkyl (such as CF₃, C₂F₅, C₃F₇, etc.), or optionally substituted phenyl. In some embodiments, X is a halogen such as F, Cl, Br, or I; CF₃SO₃—, or Ph¹-SO₃—. Ph and Ph¹ are independently optionally substituted phenyl, such as phenyl having 0, 1, 2, 3, or 4 substituents independently selected from: R′, —OR′, —COR′, —CO₂R′, —OCOR′, —NR′COR″, CONR′R″, —NR′R″, F; Cl; Br; I; nitro; CN, etc., wherein R′ and R″ are independently H, optionally substituted phenyl, or C₁₋₆ alkyl, such as methyl, ethyl, propyl isomers, cyclopropyl, butyl isomers, cyclobutyl isomers, pentyl isomers, cyclopentyl isomers, hexyl isomers, cyclohexyl isomers, etc.; C₁₋₆ alkoxy, such as methoxy, ethoxy, propoxy isomers (e.g. isopropoxy, n-propoxyl, etc.), cyclopropoxy, butoxy isomers, cyclobutoxy isomers (such as cyclobutoxy, methylcyclobutoxy, etc.), pentoxy isomers, cyclopentoxy isomers, hexoxy isomers, cyclohexoxy isomers, etc.

With respect to Formula 1, Formula 1a, Formula 1b, Formula 1c, Formula 2 and Formula 3, b is 0, 1, 2, or 3.

With respect to Formula 2 and Formula 3, a is 0, 1, 2, or 3.

In some embodiments the ketone is further represented by Formula 4:

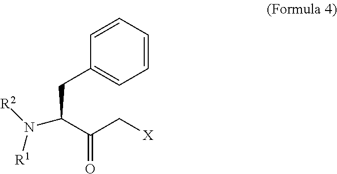

With respect to Formula 4, R², R¹, and X are the same as those described with respect to Formula 2 and Formula 3 above.

In some embodiments, the ketone is compound 7 or compound 10. These ketones may be converted to the alcohols which are compounds 107 and 110 respectively. Thus, in some embodiments, the product alcohol comprises compound 107 or compound 110.

With respect to the alcohols of Formula 3, several diastereomers are possible since there are at least two stereocenters. In some embodiments, the product alcohol, or alternatively, the alcohol of Formula 3, comprises at least one of diastereomer 1 and diastereomer 2:

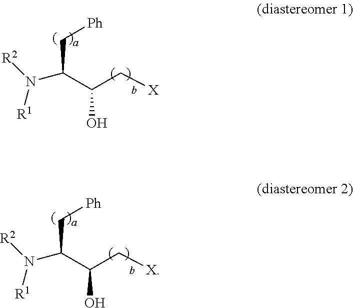

With respect to diastereomer 1 and diastereomer 2, R¹, R², Ph, X, a, and b are the same as those described with respect to Formula 3 above. In some embodiments, the ratio of diastereomer 1 to diastereomer 2 is at least about 0.5, about 0.9, about 1, or about 1.1 up to about 2, about 5, about 10, about 100, about 10000, or about 10,000.

The catalyst for the reaction may be any aluminum alkoxide, such as) Al(OR^{o})₃, wherein each R^{o} is C₁₋₆ alkyl such as methyl, ethyl, propyl isomers, cyclopropyl, butyl isomers, cyclobutyl isomers, pentyl isomers, cyclopentyl isomers, hexyl isomers, cyclohexyl isomers, etc.; or optionally substited aryl such as optionally substituted phenyl In some embodiments, each R^{o} is C(CH₃)₃. In some embodiments, R^{o} may be isopropyl or t-butyl. In some embodiments, the catalyst may be Al[OC(CH₃)₃].

The reactant alcohol may be any alcohol which may be useful as a hydride source, such an alcohol which comprises a carbon atom directly bonded to both a hydroxyl group and a hydrogen atom. The term directly bonded refers a bond is formed between the two groups without any intervening atoms, such as:

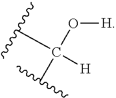

Examples of useful reactant alcohols may include methanol, ethanol, n-propyl alcohol, isopropyl alcohol, n-butanol, sec-butanol, or isobutanol etc.

In some embodiments, the reaction is carried out in the presence of an aprotic solvent. In some embodiments, a sufficient amount of the polar aprotic solvent may be used so as to interfere with hydrogen bonding between the catalyst and the ketone, and thus increase the amount of diastereomer 1 to diastereomer 2. For example, the volume ratio of the polar aprotic solvent to the reactant alcohol may at least about 1:1, about 2:1, or about 5:1, up to about 10:1 or about 100:1. In some embodiments, the polar aprotic solvent comprises ethyl acetate, tetrahydrofuran, dichloromethane, toluene, an ether, or the like.

### EXAMPLES

The asymmetric Meerwein-Ponndorf-Verley (MPV) reduction of N-(tert-butyloxycarbonyl)-(3S)-3-amino-1-chloro-4-phenyl-2-butanone (1) to its corresponding alcohol products was investigated. Although the (S,S) alcohol 2 may be favored using the traditional MPV reduction [Al(OiPr)₃ in isopropanol], the procedures provided herein improve the yield of the (R,S) product 3. While not limiting any embodiments, the experiments described below may show that the diastereoselectivity induced by the α-chiral carbon of (S) configuration and the reaction rates may be dependent on the aluminum alkoxide reagent's ability to hydrogen bond with the substrate and upon the aggregation state. By replacing the N-protecting group tert-butyloxycarbonyl (boc) with a phthalimide, which is believed to be unable to or only weakly hydrogen bond with the Al(OiPr)₃, the diastereoselectivity toward (R,S) alcohol 3 may be improved from a (R,S):(S,S) ratio of about 0.06:1 to about 1.10:1. Additionally, it was observed that the rate of the MPV reaction may be significantly increased when Al(OtBu)₃ was used in place of Al(OiPr)₃. This rate enhancement was successfully applied to reduce the prochiral ketone 1 as well as acetophenone and benzaldehyde.

All solvents used in the MPV reduction were purchased as anhydrous from Aldrich and used without further purification. Starting material 1 as well as samples of 2 and 3 were prepared from known routes. Acetophenone and benzaldehyde were distilled prior to use. All other chemicals were purchased from Aldrich and used as received. ¹H, ¹³C, and ¹⁷F NMR spectra were recorded at 400 MHz in DMSO-d₆ solvent, unless otherwise noted. Elemental analyses were performed by Atlantic Microlab. HRMS experiments were performed by Georgia Institute of Technology Bioanalytical Mass Spectrometry Facility. X-ray crystallography was performed by the X-ray Crystallography Lab at Emory University. HPLC analyses were run on an Agilent 1100 series LC with the UV detector set to 210 nm. A Phenomenex Luna 5μ C18(2) reverse phase column was used in conjunction with a guard column to prevent clogging. The mobile phase was a mixture of HPLC grade CH₃CN and H₂O with a 0.1% trifluoroacetic acid buffer. MPV reductions were carried out in a 12-reaction carousel apparatus from Brinkmann with built-in temperature controller and stir plate.

### Example 1

MPV Reduction of N-(tert-butyloxycarbonyl)-(3S)-3-amino-1-chloro-4-phenyl-2-butanone (1) with Aluminum Alkoxides. Ketone 1 (0.475 g, 1.59 mmol) was stirred with Al(OiPr)₃ (0.170 g, 0.83 mmol), Al(OtBu)₃ (0.205 g, 0.83 mmol), or Al(OEt)₃ (0.135 g, 0.83 mmol) in anhydrous isopropanol (5 mL), and the mixtures were heated to 50° C. and allowed to run under argon for 2 hours. After about five minutes, a precipitate began to appear in the Al(OtBu)₃ catalyzed reactions. By comparison, a precipitate began to appear after about an hour of stirring at 50° C. with Al(OEt)₃ catalyst and after about 1.5 hours with Al(OiPr)₃ catalyst. Since the product is less soluble than the starting ketone; it is believed that rate of precipitation correlates with the rate of the reaction. The reactions were removed from heat and ethyl acetate (EtOAc) was added. The reactions were quenched with 2 M HCl (5 mL) and the organic materials were extracted into EtOAc. Each solution was sampled twice and diluted with MeOH before running on HPLC to determine the diastereoselectivity of the reaction. No significant difference in diastereoselectivity was observed for the three catalysts.

### Example 2

The MPV reduction of ketone 1 was initially performed in isopropanol at 50° C. using Al(OiPr)₃. A set of twelve identical reactions were run simultaneously and quenched at 10-minute intervals over the course of 2 hours. The same experiment was run using Al(OtBu)₃ as the catalyst with the reactions quenched every 5 minutes for 1 hour.

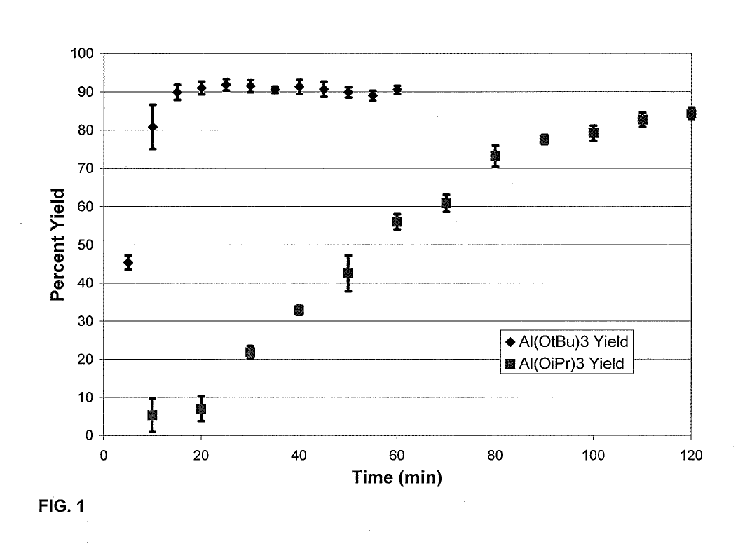

In these reactions, Ketone 1 (0.475 g, 1.59 mmol) was dissolved in anhydrous isopropanol (5 mL) at 50° C. under argon. Al(OiPr)₃ (0.170 g, 0.83 mmol) or Al(OtBu)₃ (0.205 g, 0.83 mmol) was added to start the reaction. After the specified reaction time, the reactions were removed from heat and placed in an ice bath. The reactions were quenched with 2 M HCl (2 mL) and diluted with MeOH. Each reaction mixture was sampled twice and further diluted with MeOH. These crude solutions were run directly on the HPLC without further purification. Percent conversion and yield were calculated based on calibration curves for compounds 1, 2, and 3. These kinetic experiments were run in triplicate and the results, shown in FIG. 1, demonstrate a substantial rate acceleration for the reaction when Al(OtBu)₃ is used as the catalyst as compared to when Al(OiPr)₃ is used as a catalyst. For example, the reactions with Al(OtBu)₃ achieve 90% yield in 15 minutes, while the Al(OiPr)₃ reactions reach less than 10% yield in the same time period.

### Example 3

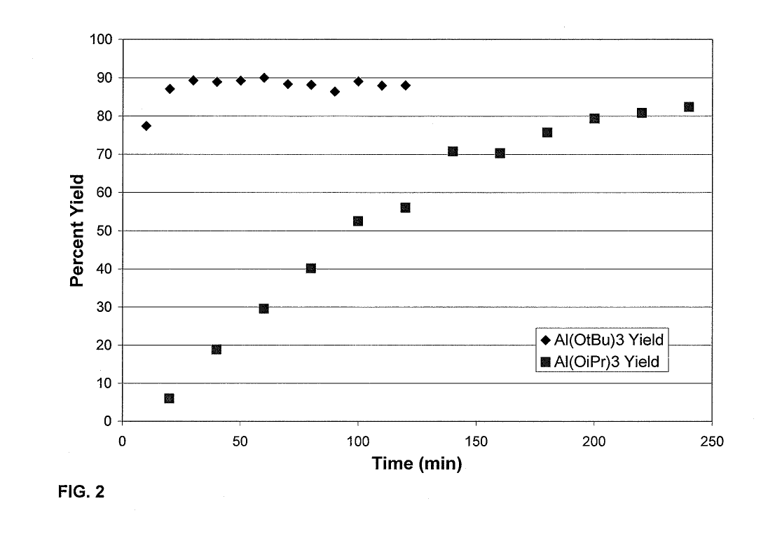

These reactions were repeated in a 9:1 toluene/isopropanol solvent mixture the procedure above was repeated except that anhydrous toluene (4.5 mL) and anhydrous isopropanol (0.5 mL) was used instead of isopropanol (5 mL). The solutions were heated to 50° C. and the reactions were allowed to run overnight (18 hours). The reaction mixtures were then removed from heat and placed in an ice bath, diluted with MeOH, and quenched with 2 M HCl (2 mL). Each solution was sampled twice and analyzed by HPLC. Al(OtBu)₃ also showed higher activity than Al(OiPr)₃ under these conditions. Because of a dilution effect, the MPV reduction in toluene required about twice as long to achieve the same product yield as the reaction in pure isopropanol (see FIG. 2).

### Example 4

The kinetics of the reduction of acetophenone was investigated. For the reduction of acetophenone to sec-phenylethanol, Al(OiPr)₃ (0.170 g, 0.83 mmol) or Al(OtBu)₃ (0.205 g, 0.83 mmol) was stirred under argon in anhydrous isopropanol (5 mL) at 50° C. Acetophenone (190 μL, 1.59 mmol) was added to start the reaction. After the allotted reaction time, each reaction was quenched with 2 M HCl (2 mL) and diluted with MeOH (10 mL). The reactions were cooled in an ice bath and sampled twice to run on the HPLC. The reactions with Al(OtBu)₃ were stopped every 15 minutes for 3 hours and the reactions with Al(OiPr)₃ ran for 7 hours with reactions stopped every 35 minutes.

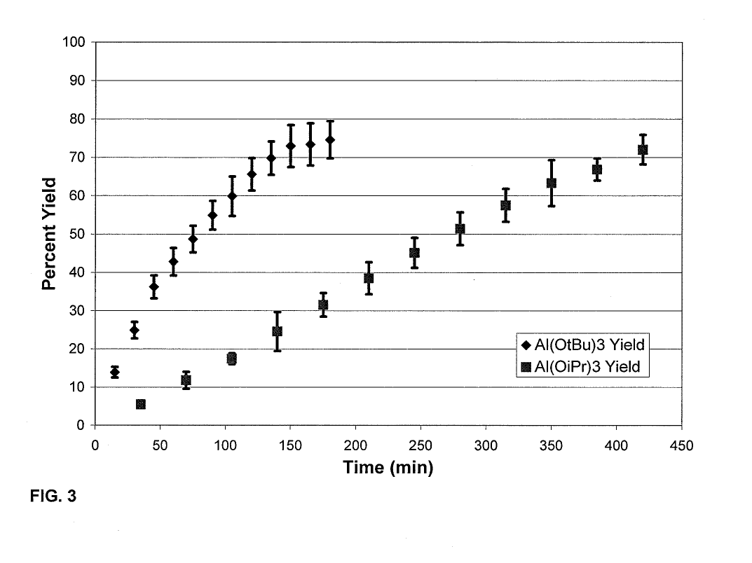

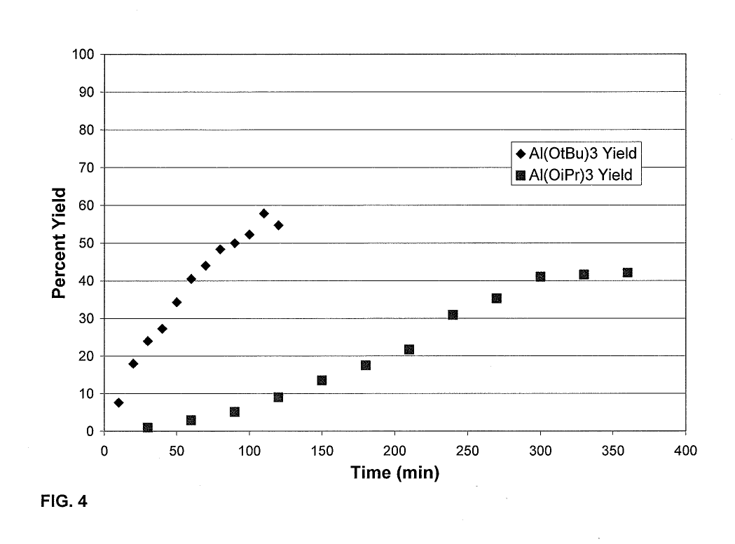

FIG. 3 shows the results of these reactions run in triplicate with the percent yield of sec-phenylethanol plotted over time. The reduction with Al(OtBu)₃ yields about 75% product after 3 hours while it takes the Al(OiPr)₃ reactions 7 hours to reach the same level of completion. A similar trend was observed when the same reactions were run in a 9:1 toluene/isopropanol solvent mixture (FIG. 4).

### Example 5

The kinetics of the reduction of benzaldehyde was investigated. Al(OiPr)₃ (0.170 g, 0.83 mmol) or Al(OtBu)₃ (0.205 g, 0.83 mmol) was stirred under argon in anhydrous isopropanol (5 mL) at 50° C. to dissolve the aluminum alkoxide. Because of the increased activity of the aldehyde, the reaction was carried out at 40° C. and stirred for at least 10 minutes before benzaldehyde (165 μL, 1.59 mmol) was added to start the reaction. After the allotted reaction time, each reaction was quenched with 2 M HCl (2 mL) and diluted with MeOH (10 mL). The reactions were cooled in an ice bath and sampled twice to run on the HPLC. Each reaction was run three times.

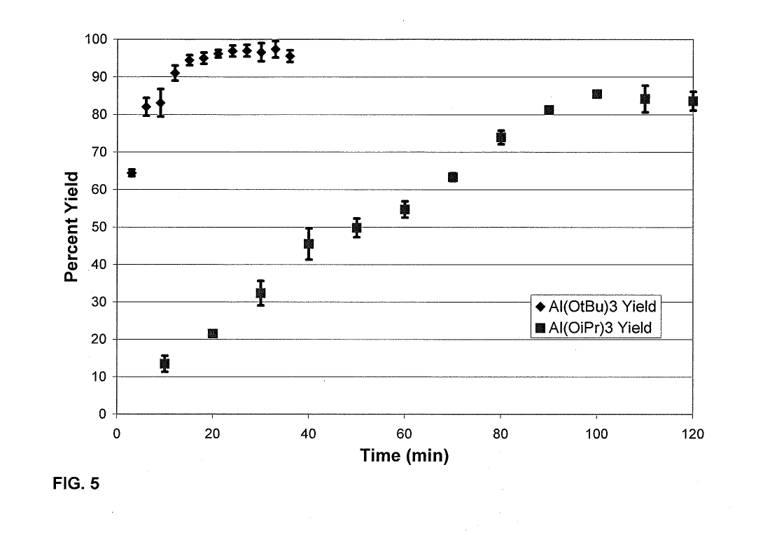

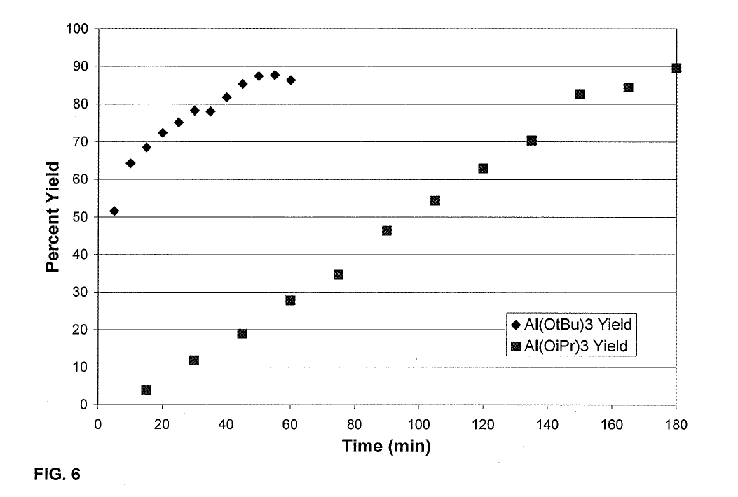

The results of the MPV reductions of benzaldehyde are shown in FIG. 5. Again, the rate increase using Al(OtBu)₃ compared to Al(OiPr)₃ is evident from this data. The reduction using Al(OtBu)₃ reaches over 80% yield in less than 10 minutes, whereas the Al(OiPr)₃ reaction requires 90 minutes to achieve the same yield. In addition, the reaction in 9:1 toluene/isopropanol takes three times as long when the catalyst is Al(OiPr)₃ instead of Al(OtBu)₃ (FIG. 6). These experiments show that the rate enhancement of Al(OtBu)₃ over Al(OiPr)₃ applies to aldehyde as well as ketone starting materials.

Thus, in some embodiments, the Al(OtBu)₃ catalyst provides a MPV reduction with an increased reaction rate. In some embodiments, increasing the rate of the reaction may not only decreases the time it takes to produce a desired compound but may also lowers the energy costs associated with heating a large vessel over a longer period of time.

### Example 6

The MPV reduction was conducted in various organic solvents as described in Example 3, where the solvent of interest was used in the place of toluene. Isopropanol (10%) was added to each solvent for the reaction to proceed in a reasonable time.

Table 1 shows the results of these reactions. The (R,S)/(S,S) ratio increased when the reaction was run in aprotic polar solvents like ethyl acetate and THF. While not limiting any embodiment by theory, it is believed that the hydrogen bonding between ketone 1 and Al(OiPr)₃ may contribute to increasing the rate of reaction by keeping 1 coordinated with the aluminum center, bringing to close proximity the two reactive centers.

TABLE 1
MPV reduction of 1 in various organic solvents

| | | % | |
|---|---|---|---|
| Solvent | (R,S)/(S,S) | Conversion | % Yield |
| EtOAc | 0.24 ± 0.01 | 73 ± 1 | 58 ± 1 |
| THF | 0.13 ± 0.01 | 92 ± 5 | 85 ± 5 |
| iPrOAc | 0.09 ± 0.00 | 100 ± 0 | 91 ± 1 |
| t-BuOH | 0.08 ± 0.00 | 98 ± 1 | 85 ± 1 |
| 2- | 0.05 ± 0.00 | 98 ± 0 | 91 ± 1 |
| BuOH | | | |
| DCM | 0.05 ± 0.00 | 100 ± 0 | 95 ± 2 |
| Toluene | 0.05 ± 0.01 | 100 ± 0 | 90 ± 1 |

### Example 7

Several derivatives of ketone 1 were prepared to investigate the effect of the nitrogen protecting group on the MPV reaction products. The synthesis of a phthalimide-protected ketone 7 was initially attempted using a procedure similar to that found in the literature to synthesize 1. This method involved the reaction of N-phthaloyl-L-phenylalanine (4) with isobutylchloroformate to form a mixed anhydride intermediate. This mixed anhydride was then reacted with diazomethane and quenched with hydrochloric acid to form the chloromethyl ketone product. Unfortunately, after several attempts, the desired product could not be isolated.

The synthetic procedure was modified to use an acid chloride intermediate instead of the unstable mixed anhydride intermediate (Scheme 1). The phthalimide-protected phenylalanine 4 was reacted with oxalyl chloride to form the acid chloride intermediate (5). Acid chloride 5 was then reacted with trimethylsilyldiazomethane and quenched with hydrochloric acid to form the final product, N-phthaloyl-(3S)-3-amino-1-chloro-4-phenyl-2-butanone (7). Compound 7 was successfully isolated in 73% yield and characterized by 1H and 13C NMR, melting point, elemental analysis, and mass spectrometry, which were in agreement with literature results.

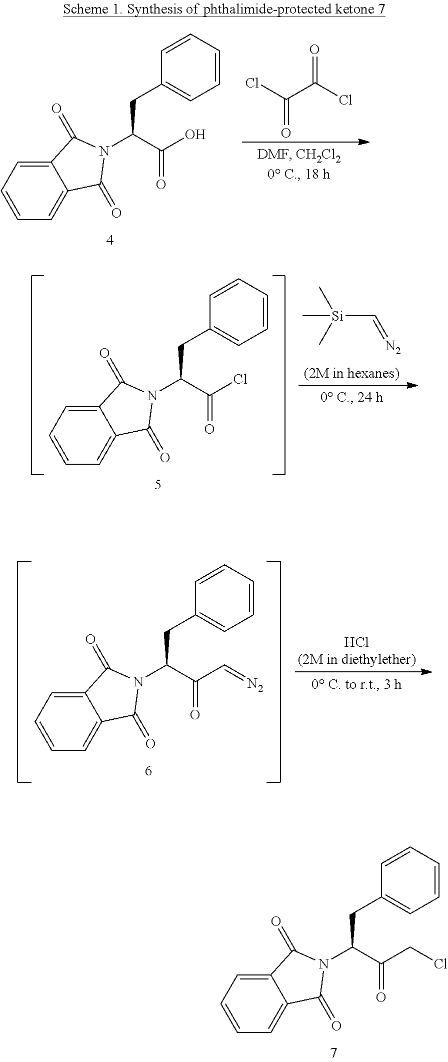

The trifluoroacetamide-protected derivative of 1, N-trifluoroacetyl-(3S)-3-amino-1-chloro-4-phenyl-2-butanone (10) was also synthesized. Following the same synthetic scheme used to synthesize compound 7, the desired product 10 was successfully isolated with a moderate 30% yield. Rather than modifying the reaction conditions, an alternative synthetic route was chosen. The free amine derivative of 1 was directly reacted with trifluoroacetic anhydride to form 10 (Scheme 2). The boc protecting group on ketone 1 was removed by treating compound 1 with hydrochloric acid to form the corresponding ammonium hydrochloride salt 8. Salt 8 was characterized by ¹H and ¹³C NMR, elemental analysis, and melting point. Hydrochloride salt 8 was then neutralized to form free amine 9. Careful attention was paid to keep compound 9 dilute and cold to prevent undesired side reactions. Trifluoroacetic acid was then added to successfully form ketone 10 in 85% yield. The product was characterized by ¹H and ¹³C NMR, melting point, elemental analysis, and mass spectrometry.

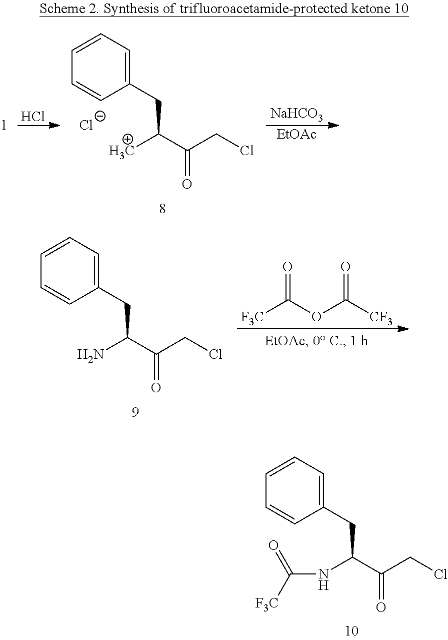

General Procedure for the Preparation of N-Protected α-Chloroketones (7 and 10). N-Protected-L-phenylalanine (5.0 mmol) was put under an argon atmosphere and anhydrous CH₂Cl₂ (25 mL) and DMF (2 drops) were added to dissolve the solid. The solution was cooled to 0° C. and oxalyl chloride (0.48 mL, 5.5 mmol) was slowly added to the solution over 15 minutes. The solution was allowed to stir at 0° C. overnight. Trimethylsilyldiazomethane (5.0 mL of a 2 M solution in hexanes, 10 mmol) was slowly added to the reaction mixture over 1 hour. The solution was allowed to stir at 0° C. under argon for 24 hours. At this time HCl (5.0 mL of a 2 M solution in diethyl ether, 10 mmol) was added. After 1 hour, the solution was allowed to warm to room temperature and the reaction was opened to the atmosphere. After 3 hours, the organic layer was washed with saturated NaHCO₃ and then with brine. The solvent was removed via rotary evaporation resulting in a yellow solid. The crude material was decolorized with charcoal in CH₃CN.

N-Phthaloyl-(3S)-3-amino-1-chloro-4-phenyl-2-butanone (7). The decolorized material was recrystallized in a 9:1 mixture of hexanes/EtOAc. Compound 7 was isolated as a white solid (1.2 g, 3.7 mmol, 73%): mp 117-118° C. (lit mp 119-120° C.); ¹H NMR δ 3.30 (br m, 2H), 4.80 (br q, 2H), 5.33 (q, 1H), 7.10 (m, 5H), 7.81 (s, 4H); ¹³C NMR δ 32.9, 47.7, 57.9, 123.4, 126.7, 128.3, 128.8, 130.9, 134.8, 136.7, 167.1, 196.9; HRMS (FAB+) calcd for C₁₈H₁₄NO₃Cl 327.0662, found 328.0731 (M+H); Elemental analysis calcd for C₁₈H₁₄NO₃Cl: C 65.96, H 4.31, N 4.27; found: C 65.90, H 4.20, N 4.21.

N-Trifluoroacetyl-(3S)-3-amino-1-chloro-4-phenyl-2-butanone (10). The ¹H NMR of the crude product showed the presence of compound 10 (˜30% yield). ¹H NMR δ 2.84 (m, 1H), 3.24 (m, 1H), 4.74 (q, 2H), 4.85 (m, 1H), 7.23 (m, 5H), 9.83 (d, 1H).

(S)-4-Chloro-3-oxo-1-phenylbutan-2-aminiumchloride (8). Ketone 1 (5.00 g, 16.8 mmol) was dissolved in EtOAc (50 mL) and cooled to 0° C. in an ice bath. Concentrated HCl (15.3 mL, 503 mmol) was added dropwise to the solution. Upon complete addition of the acid, the ice bath was removed and the solution was stirred at room temperature for 1 hour. The white solid was filtered and washed with EtOAc and Et2O to give pure salt 8 (3.85 g, 16.4 mmol, 98%): mp 170-172° C. (lit mp 170° C.); ¹H NMR δ 3.17 (m, 2H), 4.51 (s, 1H), 4.62 (2H), 7.31 (m, 5H), 8.69 (s, 3H); ¹³C NMR δ 35.0, 48.0, 57.2, 127.4, 128.9, 129.5, 134.5, 198.3; LRMS (ES+) calcd for C₁₀H₁₃NOCl₂ 234.12, found 198.2 (C₁₀H₁₃NOCl); Elemental analysis calcd for C₁₀H₁₃NOCl₂: C 51.30, H 5.60, N 5.98; found: C 54.41, H 5.62, N 6.01.

Alternative Synthesis of 10. The hydrochloride salt 8 (20 mmol) was neutralized using saturated NaHCO₃ in EtOAc. The organic layer was dried over MgSO₄ and filtered onto an ice bath. The dilute solution of the free amine was placed under an argon atmosphere and allowed to cool to 0° C. Trifluoroacetic anhydride (5.6 mL, 40 mmol) was added dropwise. The reaction mixture was poured into water (30 mL) after 1 hour and extracted with EtOAc (2×75 mL). The organic layer was washed with brine (2×30 mL), dried over MgSO₄, and concentrated under reduced pressure. Crystallization from toluene afforded the pure compound 10, which was isolated as a white solid (5.1 g, 17 mmol, 85%): mp 114-115° C. (lit mp 112° C.); ¹H NMR δ 2.85 (m, 1H), 3.25 (m, 1H), 4.72 (q, 2H), 4.83 (m, 1H), 7.25 (m, 5H), 9.81 (d, 1H); ¹³C NMR δ 34.4, 47.7, 58.3, 114.2, 117.1, 126.7, 128.3, 129.1, 136.8, 156.2, 156.5, 198.8; ¹⁷F NMR δ-75. HRMS (FAB+) calcd for C₁₂H₁₁ClF₃NO₂ 293.0430; found 294.0517 (M+H); Elemental analysis calcd for C₁₂H₁₁ClF₃NO₂: C 49.08, H 3.78, N 4.77; found: C 48.83; H 3.61; N 4.74.

Ketones 7 and 10 were reduced via the MPV procedure. Ketone 7 did not react in a pure isopropanol solution overnight at 50° C. It is believed that this may be because of ketone 7's low solubility in isopropanol. Even when acetonitrile was added as a cosolvent (1:1 acetonitrile/isopropanol), ketone 7 remained essentially insoluble and no reaction took place. At reflux in isopropanol, the solution was still heterogeneous; however, the reaction did partially proceed. About 25% of ketone 7 was converted to the corresponding alcohols with a diastereomeric ratio of about 1:1 was observed by HPLC analysis. Since the starting material readily dissolves in toluene, it was chosen as our reaction solvent with 10% isopropanol (to ensure a reasonable reaction time). The same toluene and isopropanol solvent mixture was used to reduce ketones 1, 7, and 10. Each reduction was run in triplicate overnight at 50° C. These reactions were analyzed by HPLC and the results are summarized in Table 2. The products of the reductions of 7 and 10 were also isolated and characterized by X-ray crystallography.

TABLE 2
MPV reduction of compounds 1, 7, and 10

| Reactant | (R,S)/(S,S) | % Conversion | % Yield |
|---|---|---|---|
| 1 | 0.05 ± 0.01 | 100 ± 0 | 90 ± 1 |
| 7 | 1.10 ± 0.00 | 99 ± 0 | 92 ± 0 |
| 10 | 0.03 ± 0.00 | 99 ± 0 | 99 ± 0 |

As Table 2 shows, the phthalimide-protected ketone 7 was reduced with the (R,S) diastereomers being favored with a (R,S)/(S,S) ratio of about 1.10. While not limiting any embodiment, it is believed that because ketone 7 has no hydrogen on the nitrogen atom, this may have prevented hydrogen bonding between the acidic hydrogen of the amine group and the alkoxide of the aluminum reagent. Because it is believed that the hydrogen bonding of the acidic hydrogen of the amine group helps to favor the S,S diastereomer, this may have reversed the diastereoselectivity of the MPV reaction. Thus, in some embodiments, the stereoselectivity of an MPV reduction of a ketone having an α-amine group, such as a ketone of Formula 2, may be reversed by using a protecting group which prevents the presence of a hydrogen on the amine nitrogen. For example, in some embodiments related to Formula 2, R² is not hydrogen. The reaction does not seem to be affected by the electronic effect of the protecting group on the amine (boc vs. trifluoroacetamide). Thus, it is believed that this principle may generally apply, α-amino ketones having protecting groups which allow a hydrogen on the amine nitrogen may have opposite enantioselectivity or diastereoselectivity of those which have protecting groups which do not allow a hydrogen on the amine nitrogen.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

1. A method of reducing a C═O of an aldehyde or a ketone to a CH—OH of a product alcohol comprising reacting the aldehyde or ketone in the presence of Al[OC(CH₃)₃] and a reactant alcohol which comprises a carbon atom directly bonded to both a hydroxyl group and a hydrogen atom

2. A method of reducing a C═O of an aldehyde or a ketone to a CH—OH of a product alcohol comprising reacting the aldehyde or ketone in the presence of: Al(OR^{o})₃, a reactant alcohol comprising a carbon atom directly bonded to both a hydroxyl group and a hydrogen atom, and an aprotic solvent, wherein each R^{o} is independently C₁₋₆ alkyl or optionally substituted aryl.

3. The method of claim 2, wherein the aprotic solvent comprises ethyl acetate, tetrahydrofuran, toluene, dichloromethane, or an ether.

4. The method of claim 2, wherein the volume ratio of the aprotic solvent to the reactant alcohol is at least about 1:1.

5. The method of claim 2, wherein the reactant alcohol is methanol, ethanol, n-propyl alcohol, isopropyl alcohol, n-butanol, sec-butanol, or isobutanol, and the volume ratio of the aprotic solvent to the reactant alcohol is about 5:1 to about 100:1.The method of any one of the preceding claims wherein the aldehyde or ketone is represented by a formula: 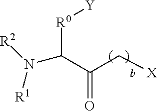
or a salt thereof;
wherein X is H, a halogen, R^{a}C(═O)O—, or R^{a}S(═O)₂O—, wherein R^{a} is C₁₋₆ alkyl, C₁₋₆F₁₋₁₃ fluoroalkyl, or optionally substituted phenyl;
Y is H, optionally substituted C₆₋₁₀ aryl, optionally substituted C₂₋₁₀ heteroaryl, halo, —OR^{b}, —SR^{b}, —NR^{b}R^{c}, —CO₂R^{f}, —OC(═O)R^{f}, —C(═O)R^{f}, —C(═O)NR^{b}R^{c}, or —NR^{b}—C(═NR^{c})—NR^{d}R^{e}, wherein R^{b}, R^{c}, R^{d}, R^{e}, and R^{f} are independently H, C₁₋₆ alkyl, or a protecting group;
R¹ is H, C₁₋₆ alkyl, COR³, or a protecting group, or R¹ and Y may together be a covalent bond connecting R⁰ to the nitrogen atom;
R² is H, C₁₋₆ alkyl, COR³, or a protecting group;
each R³ is H or optionally substituted C₁₋₁₂ hydrocarbyl;
R⁰ is C₁₋₆ alkyl or a bond; and
b is 0, 1, 2, or 3.

6. The method of claim 5, wherein the ketone is represented by a formula: 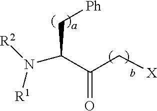
or a salt thereof;
wherein R¹ is COR³;
R² is H or COR³;
each R³ is H or optionally substituted C₁₋₁₂ hydrocarbyl; and
X is a halogen, CF₃SO₃—, or Ph¹-SO₃—;
Ph and Ph¹ are independently optionally substituted phenyl;
a is 0, 1, 2, or 3; and
b is 1, 2, or 3.

7. The method of claim 5, wherein R² is COR³.

8. The method of claim 5, wherein the ketone is further represented by a formula: 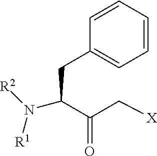
or a salt thereof

9. The method of claim 8 wherein the ketone is further represented by a formula:

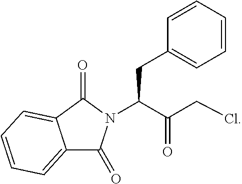 [SMILES: O=C(CCl)[C@H](Cc1ccccc1)N1C(=O)c2ccccc2C1=O]

10. The method of claim 8 wherein the ketone is further represented by a formula:

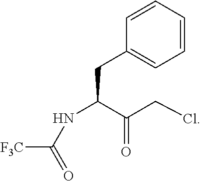 [SMILES: O=C(CCl)[C@H](Cc1ccccc1)NC(=O)C(F)(F)F]

11. The method of claim 2, wherein the product alcohol comprises at least one of diastereomer 1 and diastereomer 2: 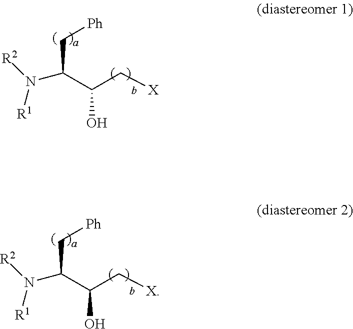
wherein R¹ is COR³;
R² is H or COR³;
each R³ is H or optionally substituted C₁₋₁₂ hydrocarbyl; and
X is a halogen, CF₃SO₃—, or Ph¹-SO₃—;
Ph and Ph¹ are independently optionally substituted phenyl;
a is 0, 1, 2, or 3; and
b is 1, 2, or 3.

12. The method of claim 11, wherein the ratio of diastereomer 1 to diastereomer 2 is at least about 0.5.

13. The method of claim 11, wherein the ratio of diastereomer 1 to diastereomer 2 has a value in the range of about 1 to about 10,000.

14. A compound represented by a formula:

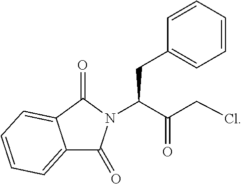 [SMILES: O=C(CCl)[C@H](Cc1ccccc1)N1C(=O)c2ccccc2C1=O]

15. A compound represented by a formula:

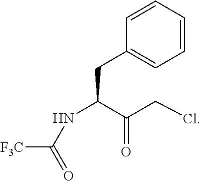 [SMILES: O=C(CCl)[C@H](Cc1ccccc1)NC(=O)C(F)(F)F]

16. A composition comprising an aldehyde or a ketone, a product alcohol, Al(OR^{o})₃, and a reactant alcohol which comprises a carbon atom directly bonded to both a hydroxyl group and a hydrogen atom;
wherein:
each R^{o} is C(CH₃)₃,
the aldehyde or the ketone and the product alcohol have identical structures except that the aldehyde or ketone has a C═O in the same position occupied by a CH—OH of the product alcohol; or
each R^{o} is C₁₋₆ alkyl; and
the ketone is represented by a formula: 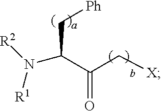
the product alcohol is represented by a formula: 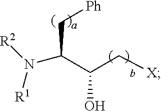
and
R¹, R², Ph, X, a, and b of the ketone are the same as R¹, R², Ph, X, a, and b of the first alcohol; wherein
R¹ is COR³;
R² is H or COR³;
each R³ is H or optionally substituted C₁₋₁₂ hydrocarbyl; and
X is a halogen, CF₃SO₃—, or Ph¹-SO₃—;
Ph and Ph¹ are independently optionally substituted phenyl;
a is 0, 1, 2, or 3; and
b is 1, 2, or 3.

17. The composition of claim 16, wherein the product alcohol comprises:

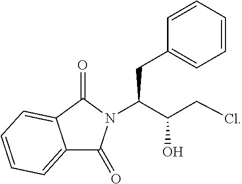 [SMILES: O=C1c2ccccc2C(=O)N1[C@@H](Cc1ccccc1)[C@@H](O)CCl]

18. The composition of claim 16, wherein the product alcohol comprises:

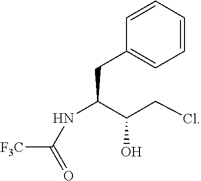 [SMILES: O=C(N[C@@H](Cc1ccccc1)[C@@H](O)CCl)C(F)(F)F]

19. A composition comprising:

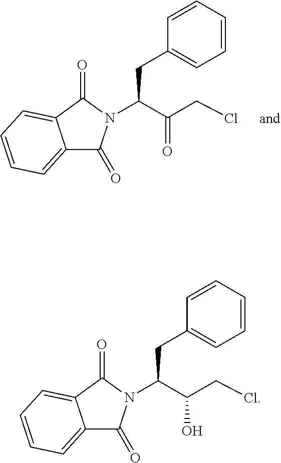 [SMILES: O=C(CCl)[C@H](Cc1ccccc1)N1C(=O)c2ccccc2C1=O ; "and" ; O=C1c2ccccc2C(=O)N1[C@@H](Cc1ccccc1)[C@@H](O)CCl]

20. A composition comprising: 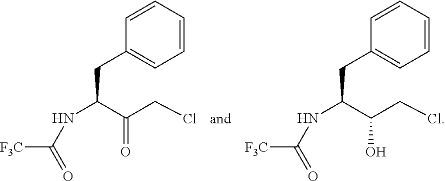 [SMILES: O=C(CCl)[C@H](Cc1ccccc1)NC(=O)C(F)(F)F ; "and" ; O=C(N[C@@H](Cc1ccccc1)[C@@H](O)CCl)C(F)(F)F]
